(19) United States
(12) Patent Application Publication
(10) Pub. No.: US 2004/0162297 A1
(43) Pub. Date: Aug. 19, 2004

(54) Adenosine derivative in polymorph I form

(76) Inventors: Paula King, Ware (GB); Barry Riddle Sickles, Durham, NC (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/481,291

(22) PCT Filed: Jun. 19, 2002
(86) PCT No.: PCT/GB02/02814

(30) Foreign Application Priority Data
Jun. 20, 2001 (GB) 0115178.6

(51) Int. Cl. A61K 31/52; C07D 473/02

(52) U.S. Cl. 514/263.2; 544/277

(57) ABSTRACT

(2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol in polymorphic form.

The present invention relates to heterocyclyl substituted adenosine derivatives. More particularly the invention is concerned with a particular physical form of (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol, pharmaceutical formulations thereof and its use in therapy.

WO99/67262 (Glaxo Group Limited) discloses certain heterocyclyl adenosine derivatives including (2S,3S,4R,5R)-2-(5-tert-butyl-[1,3,4]-oxadiazol-2-yl)-5-[6-4-chloro-2-fluorophenylamino)-9H-purin-9-yl]-tetrahydrofuran-3,4-diol, Example 14 of WO99/67262, the structure of which is indicated below as the compound of formula (A):

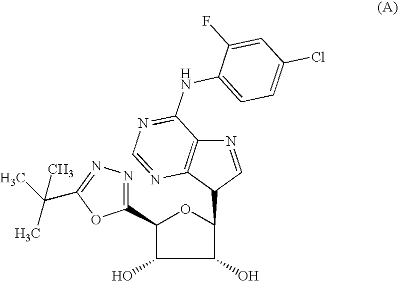

The preparation of the compound of formula (A) is described in WO99/67262. The compound of formula (A) may be prepared by the reaction of 4-chloro-2-fluoroaniline with an appropriate purinyl derivative having a suitable leaving group in the 6-position of the purine ring, optionally in the presence of a solvent at elevated temperatures. Alternatively the compound of formula (A) may be prepared by treating 9-{(3aR,4R,6S,6aR)-6-[5-tert-butyl-1,3,4-oxadiazol-2-yl]-2,2-dimethyltetrahydrofuro[3,4-d][1,3]dioxol-4-yl}-N-(4-chloro-2-fluorophenyl)-9H-purin-6-amine with trifluoroacetic,acid followed by treatment with sodium bicarbonate. Extraction of the product into ethyl acetate followed by evaporation in vacuo provides the compound of formula (A) as a buff solid.

We have now surprisingly found that the compound of formula (A) can be obtained in polymorphic forms.

There is thus provided as a first aspect of the invention (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol in polymorphic form.

We have found that the compound of formula (A) may be obtained by crystallisation under certain conditions in the form of polymorphic form I (hereinafter Polymorph I).

There is thus provided in a further aspect of the invention (2S,3S,4R,5R)-2-5-tert-butyl-[1,3,4]-oxadiazol-2-yl)-5-[6-(4-chloro-2-fluorophenylamino)-9H-purin-9-yl]-tetrahydrofuran-3,4-diol as Polymorph I.

Polymorph I exhibits particular stability at ambient temperatures, for example 15-200° C.

Polymorph I is easy to handle and particularly easy to process on a large scale and thus is useful in the preparation of pharmaceutical formulations.

In a preferred aspect the invention provides (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol in the form of Polymorph I as herein defined substantially free of any other polymorph.

In a further preferred aspect the invention (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol in the form of Polymorph I as herein defined substantially free of impurities.

By “substantially free” is meant containing less than 10%, preferably less than 5%, more preferably less than 2%, of alternative polymorph or impurity.

(2S,3S,4R,5R)-2-(5-tert-Butyl-[1,3,4]-oxadiazol-2-yl)-5-[6-(4-chloro-2-flurophenylamino)-9H-purin-9-yl]-tetrahydrofuran-3,4-diol may be prepared in polymorphic form by crystallisation of the compound under suitable conditions.

Polymorph I may be prepared substantially free from alternative polymorph by controlling crystallisation conditions.

In general, (2S,3S,4R,5R)-2-5-tert-butyl-[1,3,4]-oxadiazol-2-yl)-5-[6-(4-chloro-2-fluorophenylamino)-9H-purin-9-yl]-tetrahydrofuran-3,4-diol in the form of Polymorph I may be obtained by crystallisation of the compound by heating in N,N-dimethylformamide at a temperature sufficient to effect dissolution, for example 70-90° C., initiating crystallisation by controlled addition of water until turbidity results, and allowing to cool to ambient temperature, for example 15-25° C.

Alternatively, Polymorph I is obtained by dissolving (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol in N,N-dimethylformamide/water in a ratio of 3.5:1 to 2.5:1, preferably 3:1, optionally treating with decolourising charcoal, and cooling to less than 30° C., preferably 20-25° C., adding water and stirring the slurry prior to collecting the solid.

In a further alternative preparation Polymorph I may be prepared by dissolving (2S,3S,4R,5R)-2-(5-tert-Butyl-[1,3,4]-oxadiazol-2-yl)-5-[6-(4-chloro-2-fluorophenylamino)-9H-purin-9-yl]-tetrahydrofuran-3,4-diol in N,N-dimethylformamide and water wherein the N,N-dimethylformamide:water ratio is from 3.5:1 to 2.5:1, optionally treating with decolourising charcoal, and either cooling to less than 25° C. or cooling to less than 30° C. and seeding with polymorph I; and optionally adding toluene prior to collection of the solid.

Interconversion of one polymorph to another can occur under certain circumstances.

The methods for the preparation of polymorphic material, and in particular methods for the preparation of Polymorph I, described herein constitute further aspects of the present invention.

Polymorph I has been characterised by X-ray powder diffraction (XRPD) studies and Raman spectroscopy.

Polymorph I is characterised by having peaks in its Raman spectra at 3429, 3414 and 76 cm⁻¹.

Raman peaks are quoted to the nearest cm−1.

Polymorph I is characterised by having an XRPD pattern with signals at 4.32, 4.99, 6.23, 6.97, 8.64, 10.04, 12.53, and 14.47 (degrees 2-theta).

The skilled person will recognise that XRPD peak positions are affected by differences in sample height. The peak positions quoted herein are thus subject to a variation of +/−0.15 degrees 2-theta.

This invention further provides for a pharmaceutical composition comprising (2S,3S,4R,5R)-2-(5-tert-butyl-[1,3,4]oxadiazol-2yl)-5-[6-(4-chloro-2-fluorophenylamino)-9H-purin-9-yl]-tetrahydrofuran-3,4-diol in polymorphic form, and a pharmaceutically acceptable carrier and/or excipient.

Suitable pharmaceutically acceptable carriers and excipients are described in WO 99/967262.

(2S,3S,4R,5R)-2-(5-tert-Butyl-[1,3,4]-oxadiazol-2-yl)-5-[6-(4-chloro-2-flurophenylamino)-9H-purin-9-yl]-etrahydrofuran-3,4-diol in polymorphic form may be used for decreasing plasma free fatty acid concentration; reducing heart rate; or treating ischemic heart disease, peripheral vascular disease, stroke, pain, CNS disorder, or sleep apnoea, as described in WO 99/67262.

(2S,3S,4R,5R)-2-(5-tert-Butyl-[1,3,4]-oxadiazol-2-yl)-5-[6-(4-chloro-2-flurophenylamino)-9H-purin-9-yl]-tetrahydrofuran-3,4-diol in polymorphic form may be used in the manufacture of a medicament for use in decreasing plasma free fatty acid concentration; reducing heart rate; or treating ischemic heart disease, peripheral vascular disease, stroke, pain, CNS disorder, or sleep apnoea, as described in WO 99/67262.

WO 99/67262 (Glaxo Group Limited) is incorporated by reference herein as though fully set forth.

The following examples illustrate the invention but are not intended as a limitation thereof.

### EXAMPLES

(2S,3S,4R,5R)-2-(5-tert-Butyl-[1,3,4]oxadiazol-2-yl)-5-[6-(4-chloro-2-flurophenylamino)-9H-purin-9-yl]-tetrahydrofuran-3,4-diol was prepared according to the methods described in WO99/67262.

### Example 1

### Preparation of Polymorph I

(2S,3S,4R,5R)-2-(5-tert-Butyl-[1,3,4]oxadiazol-2-yl)-5-[6-(4-chloro-2-flurophenylamino)-9H-purin-9-yl]-tetrahydrofuran-3,4-diol (1 g) was taken up in N,N-dimethylformamide (DMF, 5 mL) and the mixture heated to 70° C. to effect dissolution. Water was added at this temperature until tubidity occurred (5 mL). The solution was then cooled to ambient (crystallisation ensued at ca. 50° C.) and allowed to stand for 1 hour before being filtered and the solid washed with water (1×2 mL). The wet cake was dried in vacuo at ambient temperature. Yield: 85%.

### Example 2

### Preparation of Polymorh I

(2S,3S,4R,5R)-2-(5-tert-butyl-[1,3,4]-oxadiazol-2-yl)-5-[6-(4-chloro-2-flurophenylamino)-9H-purin-9-yl]-tetrahydrofuran-3,4-diol (20.0 g) was dissolved in 3:1 DMF/water (266 mL), decolourising charcoal (5.0 g) added and the suspension heated at 60° C. for 1 hour. The charcoal was removed by filtration, the filter washed with 3:1 DMF/water (88 mL) and the filtrate cooled to 22-25° C. Water (44 mL) was added at 22-25° C. and the slurry stirred overnight. Water (132 mL) was added, stirring continued for 2 hours and the product collected by filtration, washed consecutively with aqueous DMF and water and then dried in vacuo at 40° C. to give Polymorph I as an off white solid (16.3 g, 81% recovery).

X-Ray Powder Diffraction

The sample preparation and acquisition conditions were as follows:

Samples were lightly ground and packed into silicon cup with a 12 mm (diameter)×0.5 mm cavity. Data were acquired using a Bruker D8 Advance X-Ray diffractometer configured with a Cu anode, primary and secondary Soller slits, secondary monochromator and scintillation counter. The generator was operated at 40 kV 40 mA. Variable divergence and antiscatter slits were set at 12 mm irradiated area, and the detector slit was set at 0.1 mm. A locked coupled step scan with 0.02 degrees 2-theta step was used. The sample was rotated.

Data obtained for Polymorph I are shown in Figure I.

Raman Spectroscopy

Raman spectra were acquired using a Nicolet 960 ESP FT-Raman spectrometer. Samples were held in glass vials; spectra of 5 different points on a sample were averaged. Data collection parameters include: Laser power: 400 mW, Resolution: 4 cm−¹, Sample gain: 1.0, Detector: InGaAs, Beamsplitter: CaF2, Correction: none, Zero filling: none, Apodization: Happ-Genzel, Phase correction: Power spectrum.

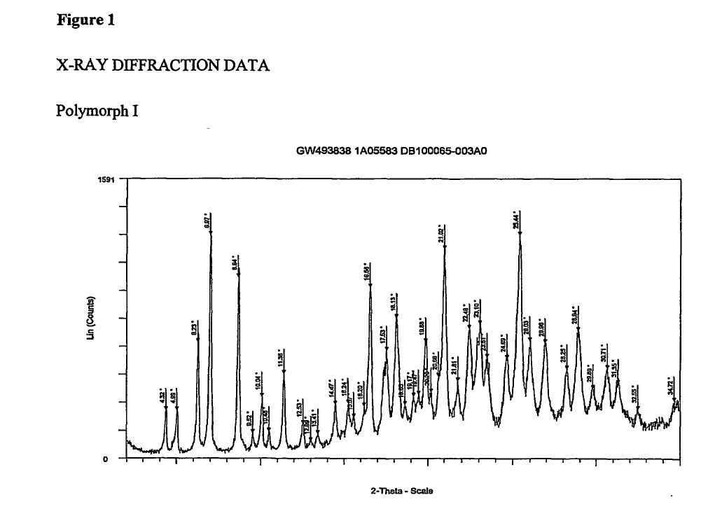

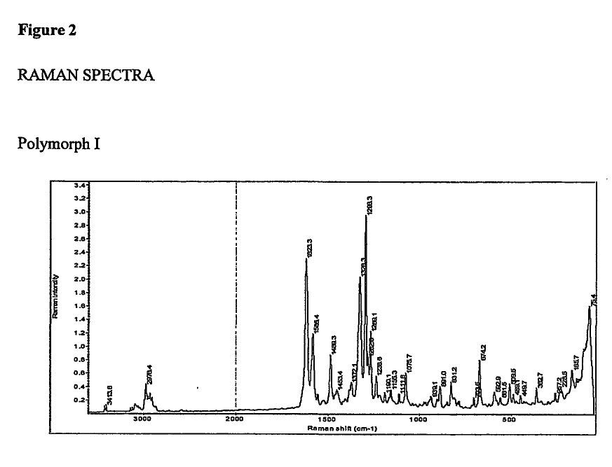

A Raman spectrum of Polymorph I are shown in FIG. 2.

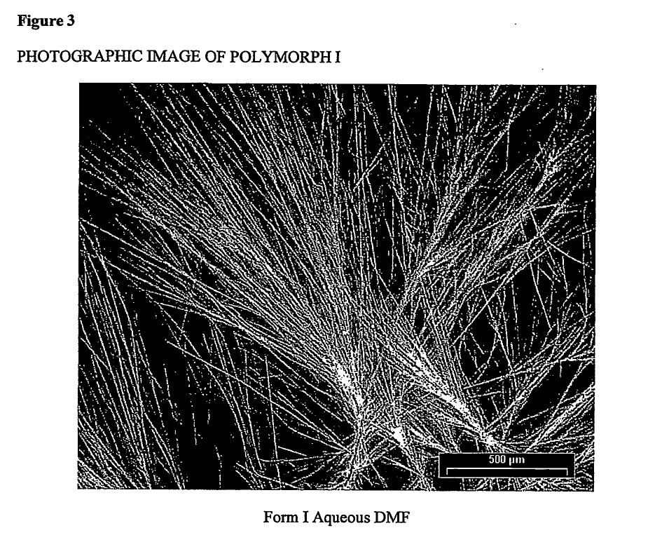

A photographic image of Polymorph I is shown in FIG. 3.

The application of which this description and these claims form a part may be used as a basis for priority in respect of any subsequent application. The claims of such subsequent application may be directed to any novel feature or combination of features relating to the invention described herein. They may take the form of product, process or use claims and may include, by way of example and without limitation, the claims that follow.

1. (2S,3S,4R,5R)-2-(5-tert-Butyl-[1,3,4]-oxadiazol-2-yl)-5-[6-(4-chloro-2-fluorophenylamino)-9H-purin-9-yl]-tetrahydrofuran-3,4-diol in polymorphic form.

2. A polymorphic form according to claim 1 wherein the polymorphic form is Polymorph I.

3. A pharmaceutical formulation comprising a polymorphic form according to claim 1 or claim 2, and a pharmaceutically acceptable carrier and/or excipient.

4. A polymorphic form according to claim 1 or claim 2 for use in decreasing plasma free fatty acid concentration; reducing heart rate; or treating ischemic heart disease, peripheral vascular disease, stroke, pain, CNS disorder, or sleep apnoea.

5. Use of a polymorphic form according to claim 1 or claim 2 in the manufacture of a medicament for use in decreasing plasma free fatty acid concentration; reducing heart rate; or treating ischemic heart disease, peripheral vascular disease, stroke, pain, CNS disorder, or sleep apnoea.

6. (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol in polymorphic form substantially as described herein in the specification and/or examples.
